(19) United States
(12) Patent Application Publication
(10) Pub. No.: US 2026/0215960 A1
(43) Pub. Date: Jul. 30, 2026

(54) Mandibular Advancement Bite Block

(71) Applicant: The Government of the United States, as Represented by the Director of the Defense Health Agency, Fort Detrick, MD (US)

(72) Inventors: Steven E. Handel, Fort Eisenhower, GA (US); Andrew S. Ryser, Fort Eisenhower, GA (US)

(21) Appl. No.: 19/457,856

(22) Filed: Jan. 23, 2026

Related U.S. Application Data
(60) Provisional application No. 63/750,494, filed on Jan. 28, 2025.

(51) Int. Cl. A61F 5/56

(52) U.S. Cl. CPC A61F 5/566

(57) ABSTRACT

A dental device that includes an adjustment device, a first body fixably coupled to the adjustment device, and a second body movably coupled to the adjustment device and spaced apart from the first body. The first body and the second body can be inserted into a patient’s mouth. The second body can move relative to the first body to adjust a position of the patient’s mandible.

### CROSS-REFERENCE TO RELATED APPLICATION

This application claims the benefit of U.S. Provisional Application No. 63/750,494, filed January 28, 2025, the entire contents of which is incorporated by reference herein in its entirety.

### FIELD

The present disclosure relates to a dental tool. More particularly, the present disclosure relates to device for adjusting a position of a patient’s mandible.

### BACKGROUND

When patients experience trauma and lose consciousness, it is important to maintain a clear airway so that the patient can continue to breathe (e.g., and avoid apneic episodes). First responders may utilize a jaw thrust method which lifts the jaw and clears the airway by moving the tongue away from the back of the throat without bending the head. Specifically, the jaw thrust moves the lower jaw (i.e., mandible) forward, which helps to push the tongue or soft tissues away, preventing airway blockage. This helps to ensure that the patient can breathe properly, allowing air to flow into the lungs. This is especially important in trauma cases, where keeping the cervical spine stable is crucial.

However, this method may be difficult to maintain because it can be physically tiring on the first responder, especially if the patient isn’t intubated right away. Therefore, an improved method for maintain an open airway in a patient is needed.

### SUMMARY

Various examples of the present disclosure can overcome various of the aforementioned and other disadvantages associated with known dental devices and offer new advantages as well.

According to one aspect of various examples of the present disclosure there is provided a dental device that includes an adjustment device, a first body fixably coupled to the adjustment device, and a second body movably coupled to the adjustment device and spaced apart from the first body. The first body and the second body can be inserted into a patient’s mouth. The second body can move relative to the first body to adjust a position of the patient’s mandible.

According to another aspect of various examples of the present disclosure, there is provided a dental device that includes a first body, a second body, a first arm fixably coupled to the first body, a second arm fixably coupled to the second body, and an adjustment device that includes an actuator. The first body includes a first channel that can receive a first number of the patient’s teeth. The second body includes a second channel that can receive a second number of the patient’s teeth. The first arm is fixably coupled to the adjustment device. The movement of the actuator permits movement of the second arm relative to the first arm.

According to another aspect of various examples of the present disclosure, there is provided a dental device that includes an adjustable first body and second body, an adjustable third body and fourth body, and a connection bar that extends between the first and third bodies. The first and second bodies can be positioned on a left side of a patient’s mouth and the third and fourth bodies can be positioned on a right side of the patient’s mouth.

According to another aspect of various examples of the present disclosure, there is provided a dental device that includes an actuator body, a first cantilever device fixed to the actuator body, and a second cantilever device movably coupled to the actuator body. The first cantilever body includes a first channel formed from a compressible material and configured to receive a first set of teeth. The second cantilever body includes a second channel formed from the compressible material and configured to receive a second set of teeth. The actuator body includes an actuator for moving the second cantilever device relative to the first cantilever device.

According to another aspect of various examples of the present disclosure, there is provided a method of adjusting a position of a patient’s mandible. The method includes inserting a first block into the patient’s mouth proximate to the maxilla. The method also includes inserting a second block into the patient’s mouth proximate to the mandible. The method further includes moving the second block relative to the first block to move the patient’s mandible relative to the maxilla.

According to another aspect of various examples of the present disclosure, there is provided a method of adjusting a position of a patient’s mandible. The method includes inserting a first channel into the patient's mouth to receive upper teeth. The method also includes inserting a second channel into the patient’s mouth to receive lower teeth. The method further includes engaging an actuator to cause the second channel to move relative to the first channel to move the patient’s mandible relative to a maxilla.

The disclosure herein should become evident to a person of ordinary skill in the art given the following enabling description and drawings. The drawings are for illustration purposes only and are not drawn to scale unless otherwise indicated. The drawings are not intended to limit the scope of the disclosure. The following enabling disclosure is directed to one of ordinary skill in the art and presupposes that those aspects within the ability of the ordinarily skilled artisan are understood and appreciated.

### BRIEF DESCRIPTION OF THE DRAWINGS

Various aspects and advantageous features of the present disclosure will become more apparent to those of ordinary skill when described in the detailed description of preferred examples and reference to the accompanying drawing wherein:

FIG. 1 is a side view of a dental device.

FIG. 2 is a lower perspective view of the dental device of FIG. 1.

FIG. 3 is a top view of the dental device of FIG. 1.

FIG. 4 is a front perspective view of the dental device of FIG. 1 inserted into a patient’s mouth.

FIG. 5 is a side perspective view of the dental device of FIG. 1 inserted into a patient’s mouth.

FIG. 6 is a top view of an alternate example of the dental device of FIG. 1.

### DETAILED DESCRIPTION

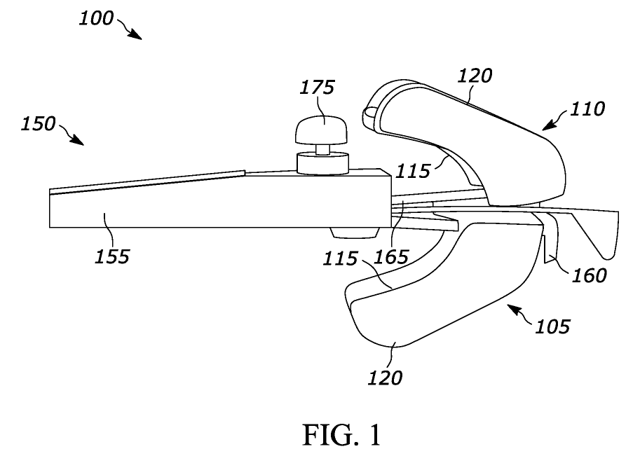

As shown in FIG. 1, the mandibular advancement device 100 includes a first or lower block or body 105 and a second or upper block or body 110, which may be at least partially spaced from the lower block 105. In the illustrated example, the lower block 105 and the upper block 110 may be substantially similar in shape and size, and description with respect to one of the blocks 105, 110 may be equally applicable to the other block 110, 105. However, other examples of the mandibular advancement device 100 have a second block 110 shaped or sized differently than the first block 105 without departing from the scope of the disclosure

In some forms, the lower and upper blocks 105, 110 may have an at least partially curved outer perimeter. For example, the lower and upper blocks 105, 110 may each include an inner edge 115 and an outer edge 120. In the illustrated example, the inner edge 115 may be at least partially curved on the lower and upper blocks 105, 110. The lower and upper blocks 105, 110 may have the same radius of curvature, although other examples may include a different radius of curvature on the different blocks 105, 110.

In some forms, the outer edge 120 may have a different curvature than the inner edge 115. For example, the illustrated outer edges 120 may be substantially linear or may have a larger radius of curvature compared to the respective inner edge 115. In other examples, the shape of the outer edge 120 on at least one of the blocks 105, 110 may be different.

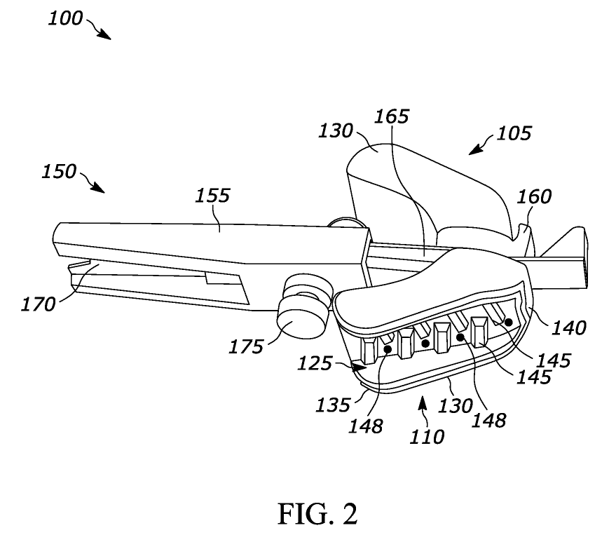

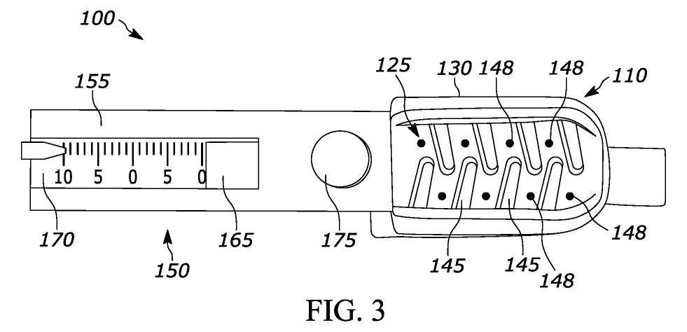

As shown in FIGS. 2 and 3, the lower and upper blocks 105, 110 may each include a channel 125 formed between a pair of spaced-apart walls 130. Each channel 125 may extend substantially along the length of the respective block 105, 110. For example, each illustrated channel 125 may extend entirely along the length of the respective block 105, 110. In this example, each channel 125 may be open on a front end and on a rear end. In other examples, one end of one or more of the channels 125 (e.g., a rear end) may be closed (e.g., include a barrier or wall).

In some forms, each channel 125 may include a substantially constant width along its length. For example, the width of each channel 125 as measured between the walls 130 may be substantially constant along the length of the channel 125.

In certain forms (see e.g., FIG. 3), the width of one or more channels 25 may taper toward the rear end of the respective block 105. For example, spacing between the illustrated walls 130 at the front end 135 may be greater than the spacing between the walls at the rear end 140.

With continued reference to FIGS. 2 and 3, the surface of the channel 125 may be constructed from a different material than the remainder of the respective block 105. For example, each block 105, 110 may be constructed from a first, rigid material (e.g., hard plastic). A second material may be applied to the inner surface of the channel 125. The second material may be more flexible than the first material. For example, the second material may be polyvinyl siloxane, or any similar rubber or silicone material.

In some forms, the channel 125 may include one or more projections 145 formed on the inner surface. For example, the illustrated channel 125 includes a series of projections 145 spaced along the length of the channel 125.

In the illustrated example, each projection 145 may be formed similar to cantilever member that extends from a wall 130 toward a center of the channel 125 along the lower surface of the channel 125 (although each projection 145 may be fixed along the lower surface). The projections 145 may be arranged to extend from alternating walls 130, although other examples may include any pattern or orientation of projections 145. In certain forms, the projections 145 may be constructed from the second material and may be flexible (e.g., compressible) when a force is applied.

In some forms, the channel 125 may include one or more openings 148 formed on the inner surface. For example, the illustrated channel 125 includes a series of openings 148 spaced along the length of the channel 125. The openings 148 may be formed at least partially through the channel 125, but not through the entirety of the respective block 105, 110. The openings 148 may be disposed in spaces between the projections 145. The openings 148 may therefore be formed in an alternating pattern opposite to the projections 145. In certain forms, the channel 125 may include only one of the projections 145 and the openings 148.

Returning to FIG. 1, the mandibular advancement device 100 may include an adjustment mechanism 150. In the illustrated example, the adjustment mechanism 150 may connect the lower and upper blocks 105, 110 together.

For example, the adjustment mechanism 150 may include a body 155 with a first projection or arm 160 that extends and connects to the lower block 105 and a second projection or arm 165 that extends and connects to the upper block 110.

As shown in FIGS. 2 and 3, the body 155 may include an indicium 170, which may take the form of a ruler. For example, the indicium 170 may provide a length measurement that can be referenced by a user.

In some forms, the second projection 165 may extend through the body 155 at least partially along the indicium 170. For example, the illustrated indicium 170 may be disposed in a groove or depression. An end of the second projection 165 may extend over the indicium 170 to provide an indication of length for the user.

In some forms, the projection 165 is substantially opaque and a user may determine a measurement based on the position of the free end of the second projection 165. In other examples, the projection 165 may be at least partially transparent or translucent to permit a user to inspect the indicium at least partially covered by the projection 165.

With continued reference to FIGS. 2 and 3, an actuator 175 may be coupled to the body 155. The illustrated actuator 175 may be a rotatable dial, although any type of actuator may be used. As described in more detail below, the actuator 175 may be used to adjust the position of the projection 165 along the indicium 170. In turn, this may adjust the position of the upper block 110 since it is rigidly connected to the projection 165.

In some forms, the actuator 175 may be selectively lockable to limit inadvertent movement. As described in more detail below, this may assist to maintain the upper block 110 in a selected position.

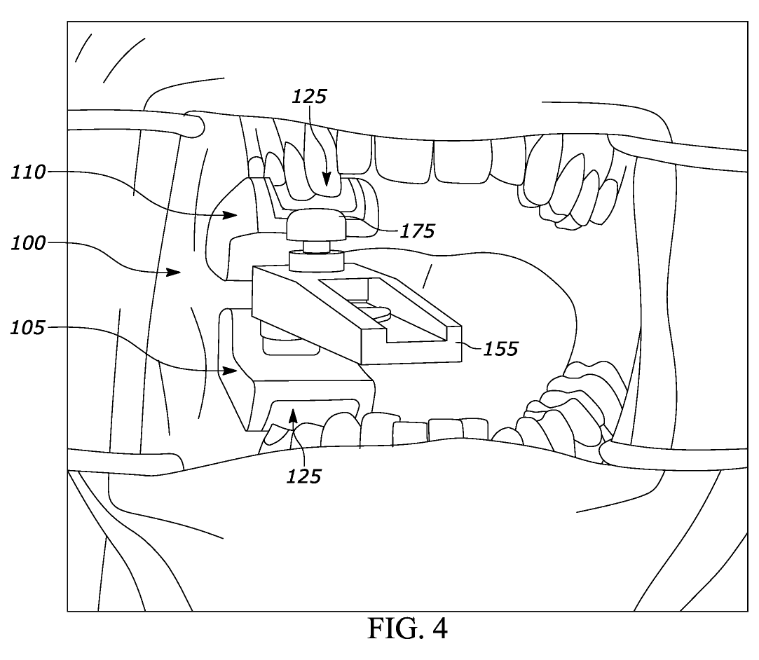

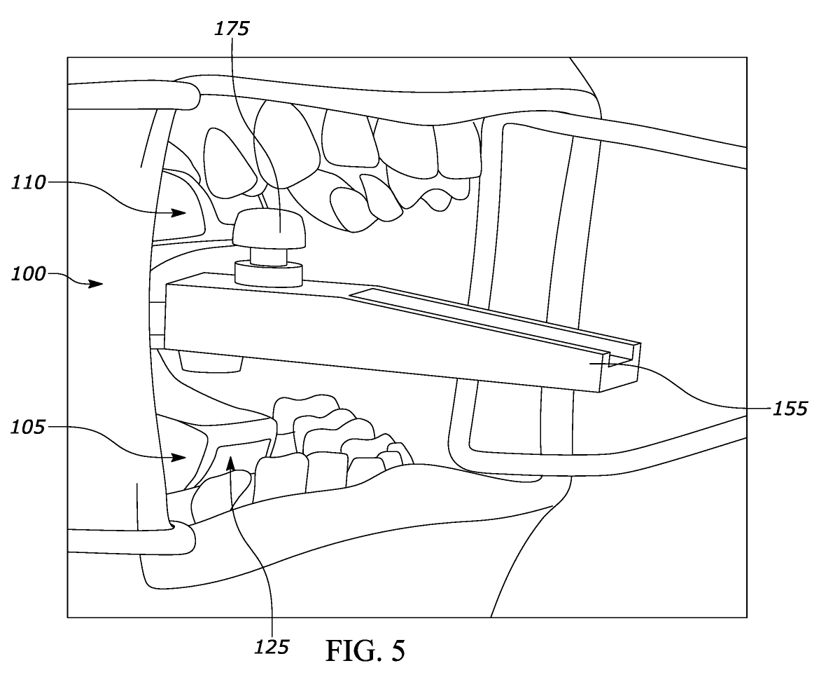

As shown in FIGS. 4 and 5, the mandibular advancement device 100 may be inserted into a patient’s mouth (e.g., where the rear end 140 of each block 105, 110 is at the back of the mouth) in use. More particularly, the mandibular advancement device 100 may be oriented so that each channel 125 on the respective block 105, 110 receives a portion of the patient’s teeth. For example, a portion of the patient’s lower teeth may be received in the channel 125 of the lower block 105 and a portion of the patient’s upper teeth may be received in the channel 125 of the upper block 110.

In some forms, the patient’s teeth may contact the second material within the respective channel 125. The second material may be at least partially compressible to conform to the shape of the patient’s teeth. Additionally, the projections 145 and/or the openings 148 may assist in supporting the teeth within the channel 125. One or more of these features may assist in maintaining the teeth within the channel 125 (e.g., limiting slipping) and/or providing a comfortable feel for the patient. In certain forms, a putty, gel, foam, paste, or other similar material may be inserted into the channel 125 prior to the patient’s teeth. The putty, gel, foam, paste may form a layer between the patient’s teeth and the second material.

In some forms, the material may be a polyvinyl siloxane (PVS) impression material that can be used to create a create a customized fit for the device. For example, the PVS may cure around a patient’s teeth to form a tighter and more comfortable fit.

When positioned within a patient’s mouth, the mandibular advancement device 100 may assist in keeping the patient’s mouth open. In other words, positioning the patient’s teeth within the channels 125 may prevent the patient’s jaws from fully closing. Keeping the patient’s jaw open may assist in providing an open airway.

Once the mandibular advancement device 100 is inserted, the user may adjust the position of the lower block 105. For example, the user may rotate the actuator 175 in a first rotational direction. Rotation of the actuator 175 may permit movement of the body relative to the projection 165 and the upper block 110 in a first linear direction. For example, the actuator 175 may include a pin pressed into the projection 165 that limits relative movement in a first position and can be rotated to move to a second position spaced apart from the projection 165 to permit relative movement. In other examples, the actuator 175 and the projection 165 may be engaged with one or more gears. Engagement of the actuator 175 may drive the projection 165 to move. In still other examples, the user may push the actuator 175 to move it from a locked position to an unlocked position. While the actuator 175 remains in the unlocked position, the user may apply a pushing or pulling force to the projection 165 to move it in the first linear direction.

Movement of the body 155 along the projection 165 in the first linear direction may cause the free end of the projection 165 to change position relative the indicium 170. The user may determine a total length of travel of the body 155 by the position of the projection 165 relative to the indicium 170.

In the illustrated example, the body 155 is fixed to the lower block 105. Therefore, movement of the body 155 in the first linear direction results in substantially the same movement of the lower block 105 in the first linear direction. When the lower block 105 is received in the patient’s mouth, the movement of the lower block 105 may cause the patients lower jaw (i.e., mandible) to move in the first linear direction. The engagement between the patient’s teeth and the second material in the channel 125 may reduce slippage and keep the mandibular advancement device 100 positioned within the patient’s mouth. In other words, the patients lower jaw may move forward as the body 155 and lower block 105 move. When the lower jaw has reached a desired position (e.g., as measured by the projection 165 and indicium 170), the user may lock the projection 165 in the selected position (e.g., using the actuator 175). This may help to maintain the patient’s lower jaw in the forward position.

In the illustrated example, movement of the first projection 160 and lower block 105 may be independent from the second projection 165 and the upper block 110. For example, upper block 110 and the second projection 165 may remain fixed or stationary within the patient’s mouth as the body 155, first projection 160, and the lower block 105 move together. This may keep the patient’s upper jaw (i.e., maxilla) fixed so that the lower jaw (i.e., mandible) can move forward. In other examples, the user may be able to adjust the position of the upper block 110 (e.g., by moving the second projection 165 in the first linear direction).

The mandibular advancement device 100 may be used to keep the patient’s mouth open and maintain an unobstructed airway (e.g., when the patient is sedated or otherwise unconscious). Movement of the lower jaw (e.g., via movement of the lower block 105) along the first linear direction may move the patient’s tongue and soft tissue away from the airway preventing obstruction of the airway. The ability to lock the lower block 105 in a desired position helps to prevent the patient’s lower jaw from slipping back to its original position (and potentially collapsing the airway) and/or requiring the user to exert energy to keep the patient’s jaw in the desired position.

When the procedure is over, the user may return the upper block 110 to its initial position by moving it in the second linear direction opposite to the first linear direction. The user may first unlock the projection 165 using the actuator 175 to permit movement along the second linear direction.

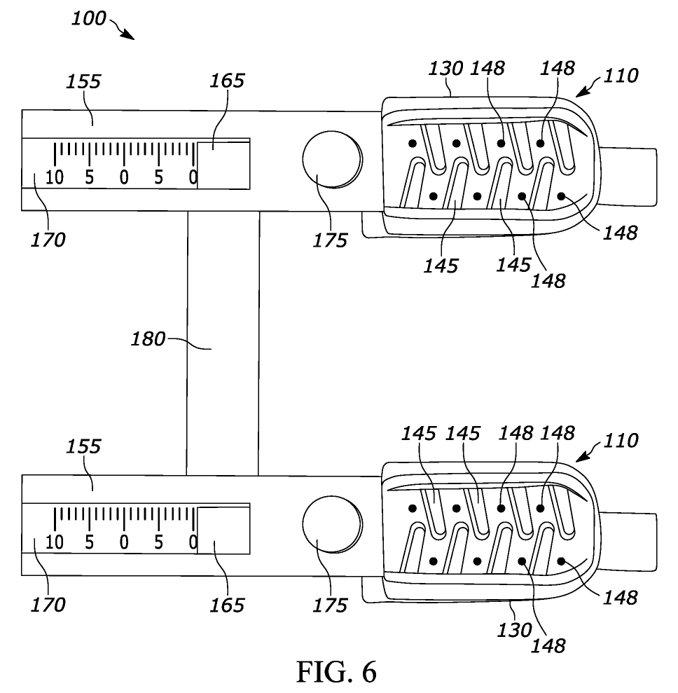

As shown in FIG. 6, an alternate example of the mandibular advancement device 100 may include a pair of lower blocks 105 and a pair of upper blocks 110 (not shown). A connection bar 180 may extend between the pair of upper blocks 110 to connect them together. Including multiple lower and upper blocks 105, 110 may permit the mandibular advancement device to contact the patient’s teeth on both sides of the mouth. In other words, the patient’s teeth on either side of their month may be received in a channel 125.

In the illustrated example, each body 155 may include an actuator 175. A clinician may adjust either actuator 175 move each of the lower blocks 105 in unison. In other examples, each actuator 175 may independently control the movement of the respective lower block 105.

One of ordinary skill will appreciate that the exact dimensions and materials are not critical to the disclosure and all suitable variations should be deemed to be within the scope of the disclosure if deemed suitable for carrying out the objects of the disclosure.

One of ordinary skill in the art will also readily appreciate that it is well within the ability of the ordinarily skilled artisan to modify one or more of the constituent parts for carrying out the various examples of the disclosure. Once armed with the present specification, routine experimentation is all that is needed to determine adjustments and modifications that will carry out the present disclosure.

The above examples are for illustrative purposes and are not intended to limit the scope of the disclosure or the adaptation of the features described herein. Those skilled in the art will also appreciate that various adaptations and modifications of the above-described preferred examples can be configured without departing from the scope and spirit of the disclosure. Therefore, it is to be understood that, within the scope of the appended claims, the disclosure may be practiced other than as specifically described.

What is claimed is:

1. A dental device comprising:
an adjustment device;
a first body fixably coupled to the adjustment device; and
a second body movably coupled to the adjustment device and spaced apart from the first body;
wherein the first body and the second body are configured to be inserted into a patient’s mouth, and wherein the second body is configured to move relative to the first body to adjust a position of the patient’s mandible.

2. The dental device of claim 1, wherein the first body includes a first channel configured to receive teeth from the patient’s upper jaw and wherein the second body includes a second channel configured to receive teeth from the patient’s lower jaw.

3. The dental device of claim 2, wherein the first body is constructed from a first material, and wherein a surface of the first channel is formed from a second material that is more flexible than the first material.

4. The dental device of claim 3, further comprising a first projection that extends from a first side wall of the first channel toward a center of the first channel and a second projection that extends from a second side wall of the first channel toward the center of the first channel, wherein the first projection and the second projection are constructed from the second material.

5. The dental device of claim 1, wherein the adjustment device includes an actuator configured to selectively limit movement of the second body relative to the first body.

6. The dental device of claim 1, wherein an arm is fixed to the second body and is received by the adjustment device, and wherein the arm is configured to move along the length of the adjustment device to change the position of the second body relative to the first body.

7. The dental device of claim 6, wherein the adjustment device includes an indicium configured to communicate a length measurement, and wherein the arm is configured to move along the indicium.

8. A dental device for adjusting a position of a patient’s mandible, the dental device comprising:
a first body including a first channel configured to receive a first number of the patient’s teeth;
a second body including a second channel configured to receive a second number of the patient’s teeth;
a first arm fixably coupled to the first body;
a second arm fixably coupled to the second body; and
an adjustment device including an actuator;
wherein the first arm is fixably coupled to the adjustment device;
wherein movement of the actuator permits movement of the second arm relative to the first arm.

9. The dental device of claim 8, wherein the first body is constructed from a first material, and wherein a surface of the first channel is formed from a second material that is more flexible than the first material.

10. The dental device of claim 9, further comprising a first projection that extends from a first side wall of the first channel toward a center of the first channel and a second projection that extends from a second side wall of the first channel toward the center of the first channel, wherein the first projection and the second projection are constructed from the second material.

11. The dental device of claim 8, wherein the adjustment device further includes an indicium formed in a groove and is configured to communicate a length measurement, and wherein the second arm is configured to move through the groove and along the indicium.

12. The dental device of claim 8, wherein the actuator includes a rotatable body that is movable from a locked position to an unlocked position, wherein the actuator is configured to contact the second arm in the locked position and is configured to be spaced apart from the second arm in the unlocked position.

13. The dental device of claim 8, wherein the first body is arranged symmetrically with respect to the second body.

14. A method of adjusting a position of a patient’s mandible, the method comprising:
inserting a first block into the patient’s mouth proximate to the maxilla;
inserting a second block into the patient’s mouth proximate to the mandible;
moving the second block relative to the first block to move the patient’s mandible relative to the maxilla.

15. The method of claim 14, further comprising moving an actuator of an adjustment device from a locked position to an unlocked position before moving the second block relative to the first block.

16. The method of claim 14, wherein the first block includes a first channel and the second block includes a second channel, the method further comprising positioning a first number of the patient’s teeth in the first channel and a second number of the patient’s teeth in the second channel.

17. The method of claim 16, wherein the first block is constructed from a first material, and wherein a surface of the first channel is formed from a second material that is more flexible than the first material.

18. The method of claim 17, further comprising first projection that extends from a first side wall of the first channel toward a center of the first channel and a second projection that extends from a second side wall of the first channel toward the center of the first channel, wherein the first projection and the second projection are constructed from the second material.

19. The method of claim 14, wherein the first block is movably coupled to an adjustment device by a first arm and the second block is fixably coupled to an adjustment device by a second arm, the method further comprising translating the second arm along a portion of the adjustment device to move the first block relative to the second block.

20. The method of claim 19, wherein the adjustment device includes an indicium configured to communicate a length measurement, the method further comprising translating the second arm along the indicium.
